SEQUENCE LISTING (4 sequences)

SEQ ID NO: 1 - protein, 463 aa, Escherichia Coli bacterium
  misc_feature (1)..(3): Xaa can be any naturally occurring amino acid
XXXMADYQGKNVVIIGLGLTGLSCVDFFLARGVTPRVMDTRMTPPXXXGLDKLPEAVERHTGSLNDEWLMAAXDLIVASPGIALAHPSLSAAADAGIEIVGDIELFCREAQAPIVAITGSNGKSTVTTLVGEMAKAAGVNVXXGVGGNIGLPAXXLMLLDDECELYVLELSSFQLETTSSLQAVAATILNVTEDHMDRYPFGLQQYRAAKLRIYENAKVCVVNADDALTMPIRGXADERCVSFGVNXXXMGDYHLNHQQGETWLRVKGEKVLNVKEMKLSGQHNYTNALAALALADAAGLPRASSLKALTTFTGLPHRFEVVLEHNGVRWINDSKATNVGSTEAALNXXGLHVDGTLHLLLGGDGKSADFSPLARYLNGDNVRLYCFGRDGAQLAALRXXPEVAEQTXXETMEQAMRLLAPRVQPGDMVLLSPACASLDQFKNFEQRGNEFARLAKELGXXXX

SEQ ID NO: 2 - protein, 463 aa, Escherichia faecalis bacterium
  misc_feature (200)..(200): Xaa can be any naturally occurring amino acid
MKKITTYQNKKVLVLGLAKSGVSAAKLLHELGALVTVNDAKQFDQNPDAQDLLTLGIRVVTGGHPIELLDEEFELIVKNPGILYTNPLVAEALTRKIPIITEVELAGQIAECPIVGITGTNGKTTTTTMIGLLLNADRTAGEARLAGNIGFPASTVAQEATAKDDLVMELSSFQLMGIETFHPQIAVITNIFEAHLDYHXGSRKEYVAAKWAIQKNMTAEDTLILNWNQVELQTLAKTTAANVLPFSTKEAVEGAYLLDGXXKLYFNEEYIMPADELGIPGSHNIENALAAICVPKLKNVSNAQIKQSLTNFSGVPHRTQFVGEVQQRRFYNDSKATNFLATEMALSXXGFDNQKLLLLAGGLDXRGNSFDELVPALLGXLKAIVLFGETKKKLAEAAKKPNIETILFAENVQTAVTIAFDYSEKDDTILLSPACASWDQYPNFEVRGEAFMQAVQQLKESEM

SEQ ID NO: 3 - protein, 463 aa, Pseudomonas aeruginosa bacterium
  misc_feature (1)..(2): Xaa can be any naturally occurring amino acid
XXMSLIASDHFRIVVGLGKSGMSLVRYLARRGLPFAVVDTRENPPELATLRAQYPQVEVRCGELDAEFLCSAXRELYVSPGLSLRTPALVQAAAKGVRISGDIDLFAREAKAPIVAITGSNAKSTVTTLVGEMAVAADKRVXXAVGGNLGTPAXXLDLLADDIELYVLELSSFQLETCDRLNAEVATVLNVSEDHMDRYXDGMADYHLAKHRIFRGARQVVVNRADALTRPLIAXDTVPCWSFGLNKPDFKAFGLIEEDGQKWLAFQFDKLLPVGELKIRGAHNYSNALAALALGHAVGLPFDAMLGALKAFSGLAHRCQWVRERQGVSYYDDSKATNVGAALAAIEGLGADIDGKLVLLAGGDGKGADFHDLREPVARFCRAVVLLGRDAGLIAQALXXGNAVPLVRVATLDEAVRQAAELAREGDAVLLSPACASLDMFKNFEERGRLFAKAVEELAXXXX

SEQ ID NO: 4 - protein, 463 aa, Staphylococcus aureus bacterium
  misc_feature (73)..(73): Xaa can be any naturally occurring amino acid
MLNYTGLENKNVLVVGLAKSGYEAAKLLSKLGANVTVNDGKDLSQDAHAKDLESMGISVVSGSHPLTLLDNNXPIIVKNPGIPYTVSIIDEAVKRGLKILTEVELSYLISEAPIIAVTGTNGKTTVTSLIGDMFKKSRLTGXXRLSGNIGYVASKVAQEVKPTDYLVTELSSFQLLGIEKYKPHIAIITNIYSAHLDYHXENLENYQNAKKQIYKNQTEEDYLICNYHQRQVIEXSEELKAKTLYFSTQQEVDGIYIKDGXXFIVYKGVRIINTEDLVLPGEHNLENILAAVLACILAGVPIKAIIDSLTTFSGIEHRLQYVGTNRTNKYYNDSKATNTLATQFALNXXSFNXQPIIWLCGGLDXRGNEFDELIPYMENXVRAMVVFGQTKAKFAKLGNSXQGKSVIEANNVEDAVDKVQDIIEPNDVVLLSPACASWDQYSTFEERGEKFIERFRAHLPSYX

(19) United States
(12) Patent Application Publication
(10) Pub. No.: US 2005/0287615 A1
(43) Pub. Date: Dec. 29, 2005

(54) Screening assay to identify inhibitors of the murd enzyme using an activator-independent murd enzyme

(75) Inventors: Haris Jahic, Waltham, MA (US); Gunther Kern, Waltham, MA (US)

Correspondence Address: FISH & NEAVE IP GROUP;ROPES & GRAY LLP, ONE INTERNATIONAL PLACE, BOSTON, MA, 02110-2624, US

(73) Assignee: AstraZeneca AB, Sodertalje (SE)

(21) Appl. No.: 10/531,844

(22) PCT Filed: Oct. 22, 2003
(86) PCT No.: PCT/GB03/04592

§ 371 Date: Apr. 18, 2005

(30) Foreign Application Priority Data
Oct. 26, 2002 (GB) 0224997.7

(51) Int. Cl. G01N 33/53; G01N 33/537; G01N 33/543

(52) U.S. Cl. 435/7.92

(57) ABSTRACT

A method of identifying inhibitors of an activator-independent MurD enzyme comprising contacting the enzyme with a test compound in the presence of enzyme substrates and appropriate buffers and detecting any modulation of enzyme activity by the test compound.

The present invention relates to improved screening assays and in particular to the use of activator-independent forms of the murein biosynthesis enzyme MurD, such as from Enterococcus faecalis (E. faecalis). Such screening assays are used to identify and characterize modulators of the MurD enzyme.

Interest has been shown in the murein (Mur) biosynthesis pathway in bacteria, this is a key component of bacterial cell wall synthesis. Enzymes in this pathway are potential targets for broad-spectrum and selective antibacterial agents.

The bacterial enzyme MurD (UDP-N-acetylmuramyl-L-alanine:D-glutamate ligase catalyses the attachment of D-glutamate to a cytoplasmic peptidoglycan precursor, UDP-N-acetylmuramyl-L-alanine. This reaction results in the formation of a peptide linkage between the amino function of D-glutamate and the carboxyl terminus of UDP-N-acetylmuramyl-L-alanine. A stoichiometric comsumption of ATP supplies the energy needed for this peptide bond formation resulting in generation of ADP and orthophosphate.

Walsh et al (Journal of Bacteriology, September 1999, 181, No. 17, 5395-5401) have examined the biochemical properties of the Mur D enzyme from two gram-negative bacteria, i.e. Escherica coli, and Haemophilus influenzae, and two gram-positive bacteria i.e. Enterococcus faecalis and Staphylococcus aureus. They established data regarding the biochemical properties of these enzymes and discussed similarities and differences between them, in particular with regard to salt-activation of the gram-negative bacteria. They report that the differences observed between the gram-positive and the gram-negative bacteria indicate that the two gram-negative bacteria may apply a more stringent regulation of cell wall biosynthesis at the early stage of the peptidogylcan biosynthesis pathway than do the two gram-positive bacteria. However, the skilled reader is aware that the substrate purification procedure used by Walsh et al cannot remove all salts that may function as enzyme activators. Therefore it is not possible to draw meaningful conclusions as to the salt-dependenc or lack of salt dependence of the two gram-positive bacteria

Indeed, we have now found that the MurD enzyme from the gram-positive bacterium Staphylococcus aureus is also salt-activated. This led to our discovery that the E. faecalis MurD enzyme has unique properties which make it possible to devise improved screening assays using an activator-independent MurD enzyme.

Therefore in a first aspect of the present invention, we provide the use of an activator-independent MurD enzyme in a screening assay to identify inhibitors of the enzyme, which assay comprises contacting the enzyme with a test compound in the presence of an enzyme substrate and appropriate buffers and detecting any modulation of enzyme activity by the test compound.

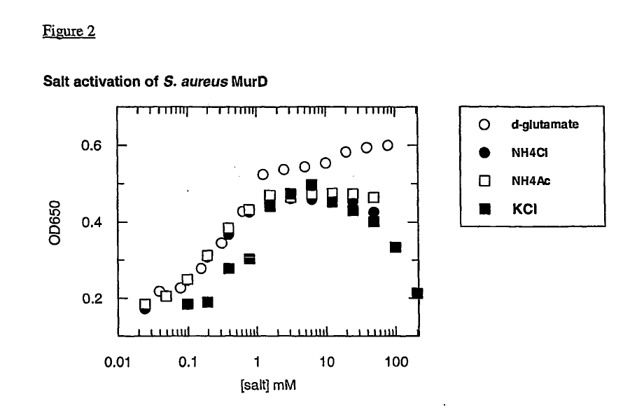

By “activator-independent” we mean that the Mur D enzyme is not activated by salt species normally associated with the substrate (here D-glutamic acid or more preferably UDP-N-acetylmuramyl-L-alanine) or other assay components. Monovalent cations such as ammonium (NH4⁺) and potassium (K⁺) are particular salt species that activate Mur D. We note that if different amounts of substrate are used in an assay e.g. for Kₘ determinations or mode of inhibition studies, the amount of the activating cation such as ammonium ions is not constant. It can therefore not be clearly identified if an activity increase is due to activation or due to an increase of substrate concentration. Contrary to the results reported by Walsh et al. we have found that the Staphylococcus aureus Mur D enzyme is activated by cations such as NH4⁺ and K⁺ (cf. FIG. 2).

Our analysis shows that activator-dependent forms of the MurD enzyme have the following common amino acid residues i.e. G96, A112, A116, V126, L129, M133, G296, P298 and V422. The indicated positions are based on the E. faecalis Mur D sequence as set out in FIG. 8 (and corresponding sequence alignments).

Therefore in a further aspect of the invention we provide the use of an activator-independent MurD enzyme which contains a MurD amino acid sequence wherein one or more of the amino acid residues at the positions given above is not as indicated for that particular amino acid. More conveniently, at least two, or at least three, or at least four, or at least five, or at least six, or at least seven, or at least 8, or all nine of the amino acid residues are not as indicated.

The activator-independent MurD may conveniently contain an amino acid sequence comprising one of more of the following residues i.e. K96, C112, G116, T126, M129, L133, N296, S298 and I422. More conveniently, at least two, or at least three, or at least four, or at least five, or at least six, or at least seven, or at least 8, or all nine of the amino acid residues are as indicated.

The activator-independent MurD enzyme conveniently comprises the following E. faecalis amino acid sequence
MKKITTYQNK KVLVLGLAKS GVSAAKLLE LGALVTVNDA KQFDQNPDAQ DILTLGIRVV TGGHPIELLD EEFEUVKNP GIPYTNPLVA EALTRKIPII TEVELAGQIA ECPIVGITGT NGKTTTTTMI GLLLNADRTA GEARLAGNIG FPASTVAQEA TAKDDLVMEL SSFQLMGIET FHPQIAVITN IFEAHLDYHG SRKEYVAAKW AIQKNMTVED TLILNWNQVE LQTLAKTTAA NVLPFSTKEA VEGAYLLDGK LYFNEEYIMP ADELGIPGSH NIENALAAIC VAKLKNVSNV QIRQTLKNFS GVPHRTQFVG EVQQRRFYND SKATNILATE MALSGFDNQK LLLLAGGLDR GNSFDELVPA LLGLKAIVLF GETKEKLAEA AKKANIETIL FAENVQTAVT LAFDYSEKDD TILLSPACAS WDQYPNFEVR GEAFMQAVQQ LKESEM
   or a Mur D amino acid sequence having at least 85% homology, such as 90 or 95% homology, therewith.

Alternatively the activator-independent Mur D amino acid sequence has at least 70% sequence identity with the above amino acid sequence.

Whilst we do not wish to be limited by theoretical considerations we believe that the activator-independent Mur D enzyme may have up to 16, such as up to 16, 14, 12, 10, 8, 6, 4, or 2, amino acids removed from the N-terminus and/or up to 12, such as up to 12, 10, 8, 6, 4, or 2, amino acids removed from the C-terminus of the enzyme.

Activator-independent Mur D sequences that do not correspond to published Mur D enzyme sequences are novel and represent a further aspect of the present invention.

Any convenient screening assay format may be used. By way of non-limiting example we disclose the following:

The enzyme substrates are conveniently UDP-MurNac-L-Ala, D-Glutamate and ATP.

The enzyme is conveniently pre-incubated with the test compound to allow inhibitors to bind to the enzyme. This may allow the detection of inhibitors with a slow binding mode to the enzyme or allow detection of specifically modifying inhibitors that may be out-competed by the substrates.

Any appropriate buffer can be used that has a pKa in the pH range where E. faecalis MurD is active (pH 7.0-10.0) Examples of convenient buffers include buffers that do not contain phosphate such as GOOD Buffer i.e. Tris or Hepes (Good, et al. (1966) Biochemistry, 5, 467477). Modulation of enzyme activity may be detected using any convenient detection system, such as those which include a colour change eg. using malachite green. These include absorbance spectrophotometers, absorbance plate reader or any other instrument that can determine the absorption of a solution between for example a wavelength range of 400 to 800 nm

Modulation of enzyme activity can be inhibition or activation of enzyme activity, conveniently enzyme inhibition.

Appropriate control reactions are conveniently performed to determine if a chemical compound interferes with the detection system and/or has an absorbance at the detection wavelength.

The test compound is any convenient compound that may be useful in pharmaceutical research. It may be a polypeptide of equal to or greater than, 2 amino acids such as up to 6 amino acids, up to 10 or 12 amino acids, up to 20 amino acids or greater than 20 amino acids such as up to 50 amino acids. For drug screening purposes, preferred compounds are chemical compounds of low molecular weight and potential therapeutic agents. They are, for example of molecular mass less than about 1000 Daltons, such as less than 800, 600 or 400 Daltons. If desired, the test compound may be a member of a chemical library. This may comprise any convenient number of individual members, for example, tens to hundreds to thousands to millions etc., of suitable compounds, for example, peptides, peptoids and other oligomeric compounds (cyclic or linear), and template-based smaller molecules, for example, benzodiazepines, hydantoins, biaryls, carbocyclic and polycyclic compounds (eg. naphthalenes, phenothiazines, acridines, steroids etc.), carbohydrate and amino acids derivatives, dihydropyridines, benzhydryls and heterocycles (eg. triazines, indoles, thiazolidines etc.). The numbers quoted and the types of compounds listed are illustrative, but not limiting. Preferred chemical libraries comprise chemical compounds of low molecular weight and potential therapeutic agents.

In a further aspect of the invention we provide a MurD enzyme modulator resulting from use of the assay method of the invention.

The activator-independent MurD enzyme may be produced using known recombinant techniques for cloning and expression (cf. Sambrook, J., E. F. Fritsch, and T. Maniatis. 1989. Molecular cloning: a laboratory manual, 2nd ed. Cold Spring Harbor Laboratory, Cold Spring Harbor, N.Y.). Convenient expression systems for the MurD enzyme include T17 promoter-driven transcription of the MurD gene in a suitable host, more conveniently E. coli. Examples of convenient expression vectors include those with a T7 promoter and suitable cloning sites such as pET28b and pET30a (Novagen Inc. Madison Wis. USA). The E. coli host strains used for expression in such a system include those that contain the 17 RNA polymerase gene that can be induced to initiate transcription of the murD gene, more specifically BL21(DE3) and HMS174(DE3).

The invention will now be illustrated by reference to the following Specific Description and Figures wherein:

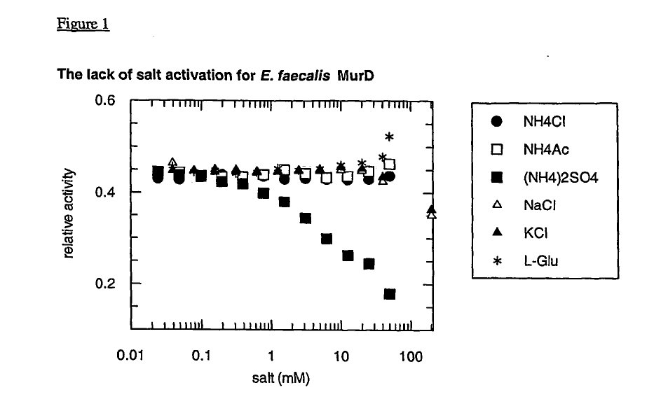

FIG. 1 shows the lack of salt activation for E. faecalis MurD and shows that all salts but SO₄-ions (=inhibitor) have no effect on E. faecalis MurD

FIG. 2 shows the salt activation of S. aureus MurD

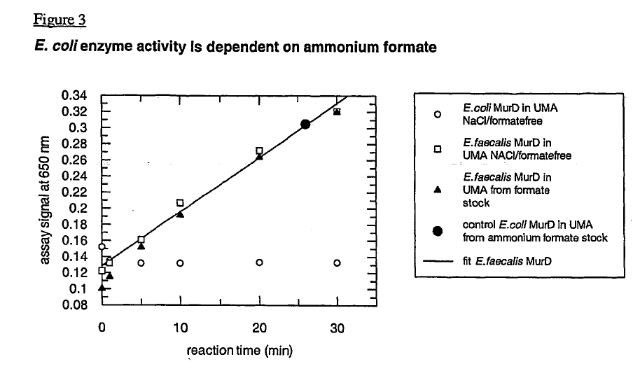

FIG. 3 shows that the E. coli enzyme activity is dependent on the presence of ammonium formate while the E. faecalis enzyme activity is independent on the presence of this salt.

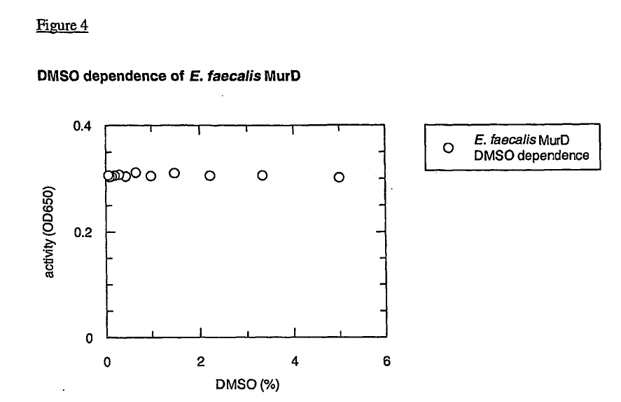

FIG. 4 shows the stability of E. faecalis Mur D in DMSO at concentrations of up to 5%.

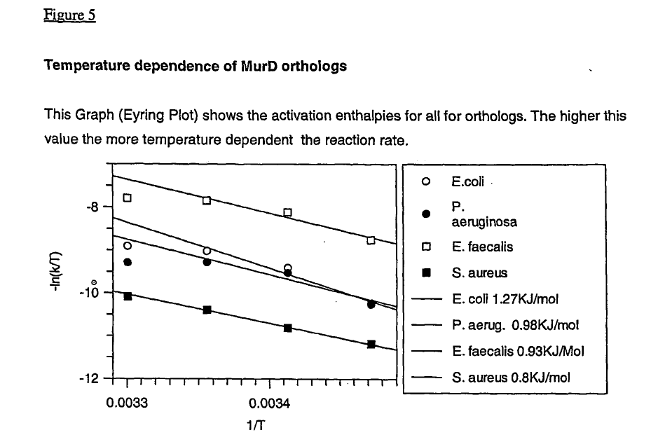

FIG. 5 shows a graph (Eyring Plot) with the activation enthalpies for several MurD orthologues, including E. faecalis. The higher this value the more temperature dependent the reaction rate

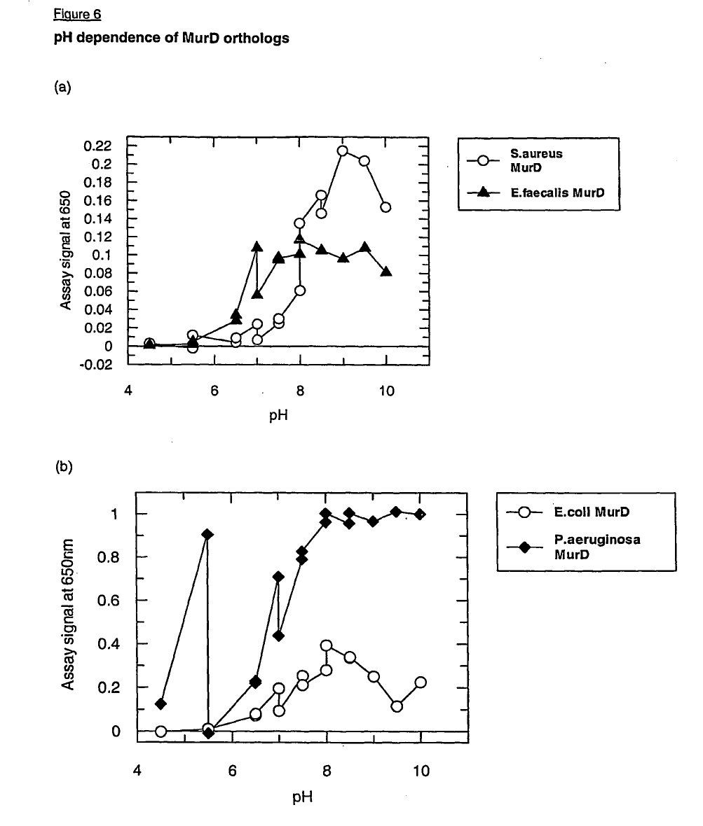

FIG. 6 (a) and (b) show the pH dependence of several MurD orthologues, including E. faecalis

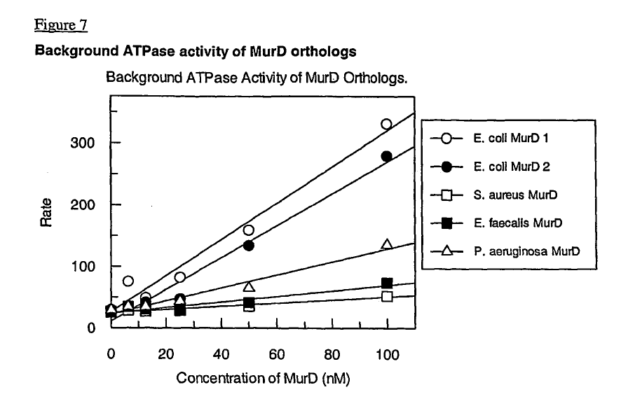

FIG. 7 shows the background ATPase activity of several MurD orthologues, including E. faecalis.

### SPECIFIC DESCRIPTION

We have established that the E. faecalis MurD enzyme has the following additional advantages for screening purposes compared to MurD enzymes from other species.
(a) it is not affected up to a dimethylsulfoxide (DMSO) concentration of 5%. Since DMSO is commonly used in screening and IC₅₀ measurements, this contributes to the stability of the assay results.
(b) its dependence of the reaction rate on temperature is low between 4° C. to 40° C. Temperature variations during assays have only a minor effect on the enzyme activity.
(c) it has the broadest pH optimum (pH 7.5-9.5). Changes in pH due to compound addition is less likely to affect activity and therefore assay results
(d) it has a low background ATPase activity. An idle ATPase activity can create a background signal in a screen (phosphate is being produced whithout catalysis occuring) so a low amount of this activity is desirable to produce a sensitive assay.

We devised a screening assay for this ortholog using Malachite Green for detection of the phosphate product. The assay includes controls to check for compound interference with the signal and interference with the detection method.
1. Compounds are preincubated with E. faecalis MurD for 5-30 min.
2. The 3 substrates ATP, UDP-MurNac-L-Ala and D-Glutamate are added to initiate the reaction.
3. After 30-60 min the reaction is stopped with a Malachite Green solution. The signal is recorded spectrophotometrically 4-10 min after the Malachite Green soltion had been added.

To control for compound interference with the assay signal, compounds are submitted to the same proceedure but in the absence of E. faecalis MurD in step 1. Compounds interfering with the signal show an increased signal relative to a control where no E. faecalis MurD was present

To control for compound interference with the Malchite Green detection method, a constant amount of phosphate (10-15 μM is replaced for E. faecalis MurD in step 1. Interference is detected by a increase or decrease of the signal relative to a control where no compound was present.

Specific assay conditions are 0.3 nM E. faecalis MurD in 50 mM Tris, 2.5 mM DTT, 10 mM MgCl₂, 0.01% Triton X-100, 50 uM ATP, 50 uM UMA, 100 uM D-Glu, pH 8.0. Typically MurD is preincubated with inhibitor for 15 min in the absence of substrates. Subsequently the reaction is initiated by adding substrates and stopped by addition of Malachite Green after 60 min. Signal is read 5 min after stopping the reaction. Although MurD activity is detected in this assay by formation of phosphate it is not limited to this detection method and can also be followed alternatively, for example, by measuring formation of other reaction products (UDP-N-acetylmuramyl-L-alanine-D-Glutamate, ADP) as well as the disappearance of the substrates (1-Glutamate, UDP-N-acetylmuramyl-L-alanine, ATP).

1. The use A method of identifying inhibitors of an activator-independent MurD enzyme comprising contacting the enzyme with a test compound in the presence of enzyme substrates and appropriate buffers and detecting any modulation of enzyme activity by the test compound.

2. The method of claim 1, wherein the activator-independent MurD enzyme comprises an amino acid sequence wherein one or more of the amino acid residues G96, A112, A116, V126, L129, M133, G296, P298 and V422 is a variant amino acid residue.

3. The method of claim 2, wherein the activator-independent MurD enzyme comprises an amino acid sequence having one or more of the amino acid residues K96, C112, G116, T126, M129, L133, N296, S298 and 1422.

4. The method of claim 1, and wherein the activator-independent MurD enzyme is an E. faecalis MurD enzyme.

5. The method of claim 1 and wherein the activator-independent MurD enzyme comprises the amino acid sequence:
MKKITTYQNK KVLVLGLAKS GVSAAKLLHE LGALVTVNDA KQFDQNPDAQ DLLTLGIRVV TGGHPIELLD EEFELIVKNP GIPYTNPLVA EALTRKIPII TEVELAGQIA ECPIVGITGT NGKTTTTTMI GLLLNADRTA GEARLAGNIG FPASTVAQEA TAKDDLVMEL SSFQLMGIET FHPQIAVITN IFEAHLDYHG SRKEYVAAKW AIQKNMTVED TLILNWNQVE LQTLAKTTAA NVLPFSTKEA VEGAYLLDGK LYFNEEYIMP ADELGIPGSH NIENALAAIC VAKLKNVSNV QIRQTLKNFS GVPHRTQFVG EVQQRRFYND SKATNILATE MALSGFDNQK LLLLAGGLDR GNSFDELVPA LLGLKAIVLF GETKEKLAEA AKKANIETIL FAENVQTAVT IAFDYSEKDD TILLSPACAS WDQYPNFEVR GEAFMQAVQQ LKESEM or a sequence having at least 85% sequence identity therewith.

6. The method of claim 1, wherein the activator-independent MurD enzyme has up to 16 amino acids removed from the N-terminus or up to 12 amino acids removed from the C-terminus of the amino acid sequence.

7. The method of claim 1, wherein the enzyme substrates are UDP-MurNac-L-Ala, D-Glutamate and ATP.

8. The method of claim 1, wherein the enzyme is pre-incubated with the test compound before contacting it with the substrates.
